(19) United States
(12) Patent Application Publication
(10) Pub. No.: US 2010/0134302 A1
(43) Pub. Date: Jun. 3, 2010

(54) SYSTEM AND METHOD FOR CONTROLLING EMOTION OF CAR DRIVER

(75) Inventors: Sung Ho Ahn, Daegu (KR); Jong Uk Kim, Daegu (KR); Kee Koo Kwon, Daegu (KR); Joon Hak Bang, Daegu (KR); Eun Ryung Lee, Daegu (KR); Jae Young Kim, Daegu (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon (KR)

(21) Appl. No.: 12/475,149

(22) Filed: May 29, 2009

(30) Foreign Application Priority Data
Dec. 1, 2008 (KR) 10-2008-0120594

(51) Int. Cl. A61B 5/18; A61B 5/02

(52) U.S. Cl. 340/576; 600/301

(57) ABSTRACT

Provided are a system and method for controlling the emotion of a car driver. A system for controlling the emotion of a car driver includes a detection unit, a control unit, and an emotion controlling unit. The detection unit detects emotion information including the voice, expression, gesture, heart rate, and temperature of the car driver. The control unit compares the detected emotion information with the prestored reference emotion information of the car driver to determine whether to control the emotion of the car driver and outputs a control signal for control of the emotion of the car driver according to the determination result. The emotion controlling unit controls the emotion of the car driver according to the control signal of the control unit.

### CROSS-REFERENCE TO RELATED APPLICATIONS

This application claims priority under 35 U.S.C. §119 to Korean Patent Application No. 10-2008-0120594, filed on Dec. 1, 2008, in the Korean Intellectual Property Office, the disclosure of which is incorporated herein by reference in its entirety.

### TECHNICAL FIELD

The following disclosure relates to a system and method for controlling the emotion of a car driver, and in particular, to a system and method for controlling the emotion of a car driver, which controls the emotion of a car driver according to the emotional state of the car driver by detecting a change in the emotion of the car driver.

### BACKGROUND

In regard to the functions of cars, many technologies or services focusing on the conveniences of car drivers have been developed, but the development of technologies or services considering the emotion of car drivers remains insufficient.

If a car driver loses his emotional balance in driving a car, he may lose his attention and instant judgment on the driving. In this case, the possibility of a traffic accident may increase significantly, and in the worst case, the possible traffic accident may kill the car driver.

For example, a car driver may drive on an expressway for a long time (especially in the night) without changing the driving speed. In this case, because the car driver may drive drowsy, he may cause a big traffic accident. As another example, a car driver may be stressed by another car driver while driving a car. In this case, because the car driver may lose his attention due to the stress, he may cause a minor traffic accident or a big traffic accident by violating the traffic lane/signal regulation.

Thus, while the performances of various safety devices installed in the car are important for the safety of the car driver, what is more important is to maintain the emotional balance of the car driver in order to prevent an otherwise-possible traffic accident.

### SUMMARY

In one general aspect, a system for controlling the emotion of a car driver includes: a detection unit detecting emotion information including the voice, expression, gesture, heart rate, and temperature of the car driver, a control unit comparing the detected emotion information with the prestored reference emotion information of the car driver to determine whether to control the emotion of the car driver and outputting a control signal for control of the emotion of the car driver according to the determination result, and an emotion controlling unit controlling the emotion of the car driver according to the control signal of the control unit.

In another general aspect, a method for controlling the emotion of a car driver includes: receiving emotion information including at least one of the voice, expression, gesture, and vital data of a car driver, comparing the received emotion information with the prestored reference emotion information of the car driver to determine whether to control the emotion of the car driver, and controlling the emotion of the car driver according to the determination result.

Other features and aspects will be apparent from the following detailed description, the drawings, and the claims.

### BRIEF DESCRIPTION OF THE DRAWINGS

FIG. 1 is a block diagram of a system for controlling the emotion of a car driver according to an exemplary embodiment.

FIG. 2 is a block diagram of a detection unit of the emotion control system according to an exemplary embodiment.

FIG. 3 is a block diagram of a control unit of the emotion control system according to an exemplary embodiment.

FIG. 4 is a flow chart illustrating a method for controlling the emotion of a car driver according an exemplary embodiment.

### DETAILED DESCRIPTION OF EMBODIMENTS

Hereinafter, exemplary embodiments will be described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience. The following detailed description is provided to assist the reader in gaining a comprehensive understanding of the methods, apparatuses, and/or systems described herein. Accordingly, various changes, modifications, and equivalents of the methods, apparatuses, and/of systems described herein will be suggested to those of ordinary skill in the art. Also, descriptions of well-known functions and constructions may be omitted for increased clarity and conciseness.

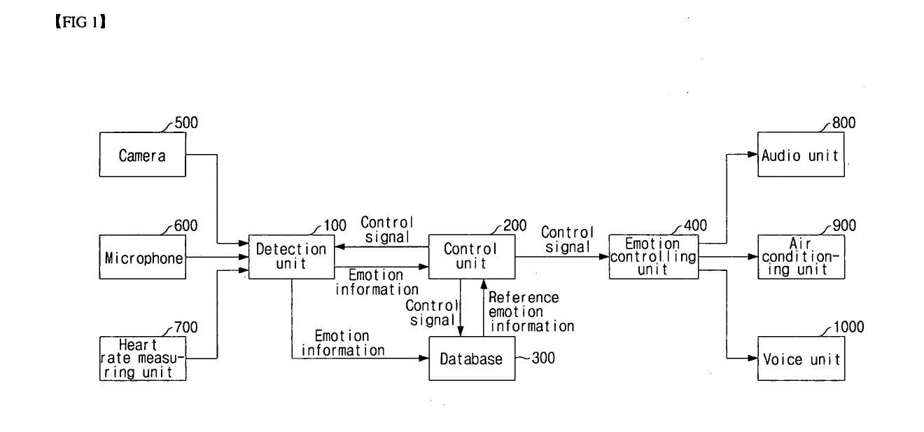

Hereinafter, a system for controlling the emotion of a car driver according to an exemplary embodiment will be described with reference to FIGS. 1 to 3.

Hereinafter, a system for controlling the emotion of a car driver will also be referred to as an emotion control system.

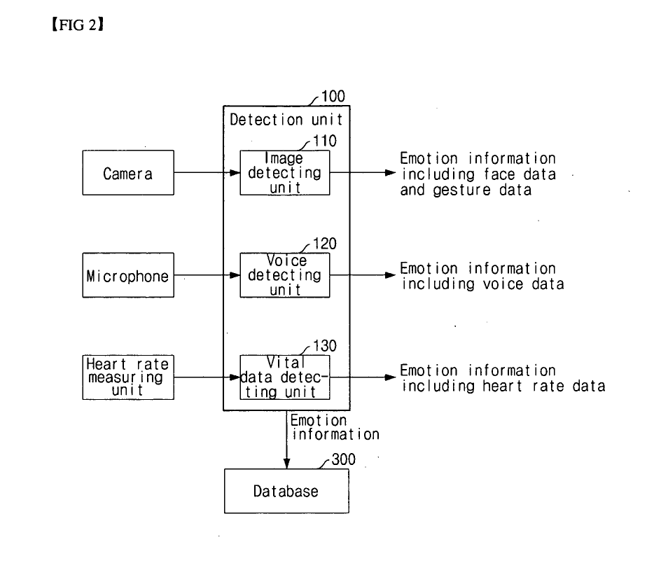

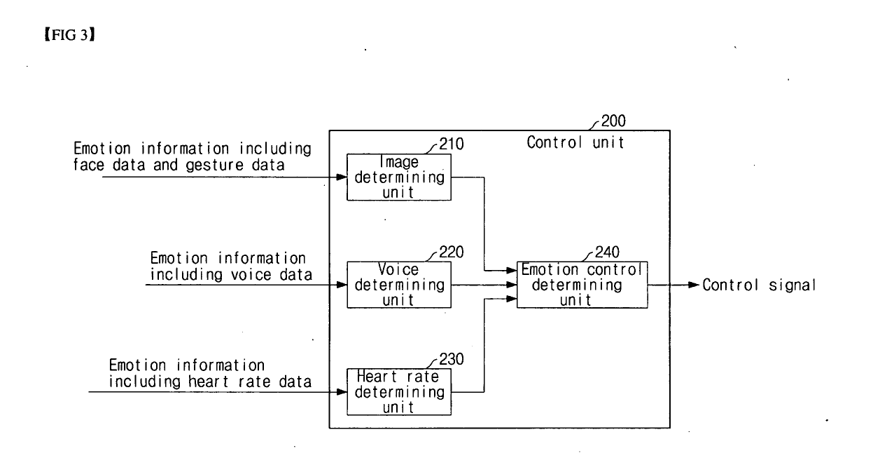

FIG. 1 is a block diagram of an emotion control system according to an exemplary embodiment. FIG. 2 is a block diagram of a detection unit of the emotion control system according to an exemplary embodiment. FIG. 3 is a block diagram of a control unit of the emotion control system according to an exemplary embodiment.

Referring to FIG. 1, an emotion control system according to an exemplary embodiment includes a detection unit 100, a control unit 200, a database 300, and an emotion controlling unit 400.

Referring to FIG. 2, the detection unit 100 includes an image detecting unit 110, a voice detecting unit 120 and a vital data detecting unit 130, and detects emotion information including the voice, expression, gesture, heart rate, and temperature of a car driver.

A camera 500 installed in the car captures an image of the motion or face of the car driver, and the image detecting unit 110 detects the gesture or expression of the car driver from the captured input image.

The voice detecting unit 120 detects the voice of the car driver through a microphone 600 installed in the car.

The vital data detecting unit 130 detects the vital data (e.g., the heart rate, temperature, and pulse) of the car driver through a heart rate measuring unit 700.

The detection unit 100 provides the detected emotion information to the control unit 200 and to the database 300.

In an exemplary embodiment, the detected emotion information may be provided directly to the database 300. In another exemplary embodiment, the detected emotion information may be provided to the database 300 through the control unit 200.

Referring to FIG. 3, the control unit 200 includes an image determining unit 210, a voice determining unit 220, a heart rate determining unit 230, and an emotion control determining unit 240. The control unit 200 compares the detected emotion information with the prestored reference emotion information of the car driver to determine whether to control the emotion of the car driver, and outputs a control signal for control of the emotion of the car driver according to the determination result.

Herein, the reference emotion information may be prestored in the database 300. In an exemplary embodiment, the reference emotion information may be average emotion data including the voice, expression, gesture, heart rate, and temperature of ordinary car drivers, which are detected when the ordinary car drivers are in a calm state. In another exemplary embodiment, the reference emotion information may be emotion data including the voice, expression, gesture, heart rate, and temperature of the car driver, which are detected when the car driver is in a calm state.

The following description will be made on the assumption that the reference emotion information is the average emotion data.

The image determining unit 210 receives the emotion information including the expression and gesture of the car driver, compares the expression and gesture included in the received emotion information with the expression and gesture included in the reference emotion information, and determines the emotional state (e.g., calm, stress, anger, bad mood, or excitation) of the car driver according to the current shapes of the face and mouth of the car driver.

The voice determining unit 220 receives the emotion information including the voice of the car driver, compares the voice included in the received emotion information with the voice included in the reference emotion information, and determines the emotional state of the car driver according to whether abuses are included in the received voice and the result of analyzing the voiceprint of the received voice.

Thus, the voice included in the reference emotion information may be data obtained by voiceprint analysis. The voice determining unit 220 may have a voice recognition technology that recognizes voices to determine whether abuses are included in the received voice. The voice recognition technology may recognize tones or volumes as well as syllables or phonemes. The heart rate determining unit 230 receives the emotion information including the heart rate of the car driver, and determines the emotional state of the car driver according to the heart rate included in the received emotion information.

For example, the heart rate determining unit 230 compares the heart rate included in the received emotion information with the heart rate included in the reference emotion information, and determines the emotional state (e.g., excitation) of the car driver according to whether the received heart rate is higher than the reference heart rate.

The emotion control determining unit 240 determines whether to control the emotion of the car driver, on the basis of the determination results of the image determining unit 210, the voice determining unit 220 and the heart rate determining unit 230.

For example, the emotion control determining unit 240 determines to control the emotion of the car driver, if at least one or more of the image determining unit 210, the voice determining unit 220 and the heart rate determining unit 230 determine that the car driver is not in a calm state.

If the emotion control determining unit 240 determines to control the emotion of the car driver, the control unit 200 controls the emotion controlling unit 400 to control the emotion of the car driver.

An emotion control index of the car driver according to the emotional state of the car driver, which is used by the control unit 200 to control the emotion controlling unit 400, and the corresponding emotion control information may be prestored as average data in the database 300.

Herein, the emotion control index may be a value corresponding to the determination results of the image determining unit 210, the voice determining unit 220 and the heart rate determining unit 230. For example, if all of the determining units determine that the car driver is in a calm state, the emotion control index is ‘0’; if one of the determining units determines that the car driver is not in a calm state, the emotion control index is ‘1’; if two of the determining units determine that the car driver is not in a calm state, the emotion control index is ‘2’; and if all of the determining units determine that the car driver is not in a calm state, the emotion control index is ‘3’.

The emotion control information may include a music play list, a humor, a joke, an accident possibility notice message, a scent spray frequency, and an oxygen generation frequency, for control of the emotion of the car driver.

For example, if the emotion control index is ‘0’ (e.g., if the car driver is in a calm state), because the control of the emotion of the car driver is not necessary, the emotion controlling unit 400 does not set the emotion control information. If the emotion control index is ‘1’ (e.g., if the car driver is relatively less stressed, excited or angered), because the control of the emotion of the car driver is necessary, the emotion controlling unit 400 sets the emotion control information. Thus, if the emotion control index is ‘1’, the music play list for control of the emotion of the car driver may be set to be ballad or classic music. If the emotion control index is ‘3’ (e.g., if the car driver is most stressed, excited or angered), the music play list for control of the emotion of the car driver may be set to be dance music for change of the mood of the car driver. The scent spray frequency, the oxygen generation frequency, the level of the joke or humor, and the accident possibility notice message may also vary depending on the emotion control index.

In an exemplary embodiment, the emotion control information may be average data obtained through the experiment on a plurality of drivers. In another exemplary embodiment, the emotion control information may be set by the car driver himself.

If the emotion control determining unit 240 determines not to control the emotion of the car driver, the control unit 200 controls the detection unit 100 to continue to detect the emotional state of the car driver, and controls the database 300 to erase the stored emotion information.

The database 300 stores the detected emotion information of the car driver and the reference emotion information that is used as a criterion for determining whether to control the emotion of the car driver.

The database 300 may also store the emotion control index of the car driver according to the emotional state of the car driver and the corresponding emotion control information, according to a method for the control unit 200 to control the emotion controlling unit 400.

In order to control the emotion of the car driver, the emotion controlling unit 400 performs at least one or more of the play of therapy music, the provision of a humor, a joke or an accident possibility notice message, the spray of scent, and the generation of oxygen.

Thus, the emotion controlling unit 400 may include an audio unit 800, an air conditioning unit 900, and a voice unit 1000 that are installed in the car.

In this way, the emotion controlling unit 400 controls the emotion of the car driver to relieve the car driver from the stress in the driving or to calm the anger of the car driver in the driving, thereby preventing an otherwise-possible traffic accident.

The emotion control system may further include a control panel (not illustrated).

For example, the control panel has a function key or button for selection of a function, and outputs a detection request signal according to the control of the function key or button by the car driver.

The detection request signal is to request the detection of the emotion information for update of the reference emotion information of the car driver.

Upon receiving the detection request signal, the control unit 200 controls the detection unit 100 to detect the emotion information including the voice, expression, gesture, heart rate, and temperature of the car driver, and controls the database 300 to store the received emotion information as the reference emotion information of the car driver in an update fashion.

Even if the controller 200 determines to perform the emotion control and controls the emotion controlling unit 400 to perform the emotion control, the car driver may think the emotion control to be unnecessary. In this case, the car driver may press a specific button (e.g., an emotion control exclusion button) of the control panel so that the control unit 200 may train the corresponding voice, expression, gesture, heart rate, and temperature of the car driver as the calm state (not the angry state) of the car driver and adjust the reference emotion information in the database 300.

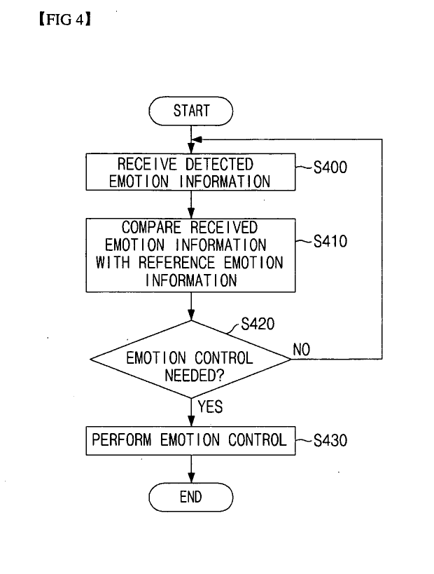

Hereinafter, a method for controlling the emotion of a car driver according to an exemplary embodiment will be described with reference to FIG. 4.

FIG. 4 is a flow chart illustrating a method for controlling the emotion of a car driver according an exemplary embodiment.

Referring to FIG. 4, in operation S400, the control unit 200 receives the detected emotion information of the car driver.

In operation S410, the control unit 200 compares the received emotion information with the prestored reference emotion information.

Herein, the reference emotion information may be prestored in the database 300. In an exemplary embodiment, the reference emotion information may be average emotion data including the voice, expression, gesture, heart rate, and temperature of ordinary car drivers, which are detected when the ordinary car drivers are in a calm state. In another exemplary embodiment, the reference emotion information may be emotion data including the voice, expression, gesture, heart rate, and temperature of the car driver, which are detected when the car driver is in a calm state.

Thus, the control unit 200 compares the voice, expression, gesture, heart rate, and temperature included in the received emotion information with those included in the prestored reference emotion information.

In operation S420, the control unit 200 determines whether to control the emotion of the car driver according to the comparison result.

For example, the control unit 200 may determine the emotional state (e.g., calm, stress, anger, bad mood, or excitation) of the car driver according to the current shapes of the face and mouth of the car driver, may determine the emotional state of the car driver according to whether abuses are included in the received voice and the result of analyzing the voiceprint of the received voice, and may determine the emotional state (e.g., excitation) of the car driver according to whether the received heart rate is higher than the reference heart rate.

If the control unit 200 determines to control the emotion of the car driver (in step S420), the control unit 200 proceeds to operation S430. In operation S430, the control unit 200 controls the emotion controlling unit 400 to perform the emotion control (e.g., at least one or more of the play of therapy music, the provision of a humor, a joke or an accident possibility notice message, the spray of scent, and the generation of oxygen).

An emotion control index of the car driver according to the emotional state of the car driver, which is used by the control unit 200 to control the emotion controlling unit 400, and the corresponding emotion control information may be prestored as average data in the database 300.

Herein, the emotion control index may be a value corresponding to the determination results of the image determining unit 210, the voice determining unit 220 and the heart rate determining unit 230. For example, if all of the determining units determine that the car driver is in a calm state, the emotion control index is ‘0’; if one of the determining units determines that the car driver is not in a calm state, the emotion control index is ‘1’; if two of the determining units determine that the car driver is not in a calm state, the emotion control index is ‘2’; and if all of the determining units determine that the car driver is not in a calm state, the emotion control index is ‘3’.

The emotion control information may include a music play list, a humor, a joke, an accident possibility notice message, a scent spray frequency, and an oxygen generation frequency, for control of the emotion of the car driver.

For example, if the emotion control index is ‘0’ (e.g., if the car driver is in a calm state), because the control of the emotion of the car driver is not necessary, the emotion controlling unit 400 does not set the emotion control information. If the emotion control index is ‘1’ (e.g., if the car driver is relatively less stressed, excited or angered), because the control of the emotion of the car driver is necessary, the emotion controlling unit 400 sets the emotion control information. Thus, if the emotion control index is ‘1’, the music play list for control of the emotion of the car driver may be set to be ballad or classic music. If the emotion control index is ‘3’ (e.g., if the car driver is most stressed, excited or angered), the music play list for control of the emotion of the car driver may be set to be dance music for change of the mood of the car driver. The scent spray frequency, the oxygen generation frequency, the level of the joke or humor, and the accident possibility notice message may also vary depending on the emotion control index.

In an exemplary embodiment, the emotion control information may be average data obtained through the experiment on a plurality of drivers. In another exemplary embodiment, the emotion control information may be set by the car driver himself.

On the other hand, if the control unit 200 determines not to control the emotion of the car driver (in step S420), the control unit 200 returns to operation S400 in order to control the detection unit 100 to detect the emotion information of the car driver.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A system for controlling the emotion of a car driver, comprising:
a detection unit detecting emotion information including the voice, expression, gesture, heart rate, and temperature of the car driver;
a control unit comparing the detected emotion information with the prestored reference emotion information of the car driver to determine whether to control the emotion of the car driver and outputting a control signal for control of the emotion of the car driver according to the determination result; and
an emotion controlling unit controlling the emotion of the car driver according to the control signal of the control unit.

2. The system of claim 1, further comprising a database storing at least one of the detected emotion information of the car driver and the reference emotion information that is used as a criterion for determining whether to control the emotion of the car driver.

3. The system of claim 1, wherein the detection unit comprises;
an image detecting unit detecting the expression and gesture of the car driver;
a voice detecting unit detecting the voice of the car driver; and
a vital data detecting unit detecting the vital data of the car driver.

4. The system of claim 1, wherein the reference emotion information is average emotion data including the voice, expression, gesture, heart rate, and temperature of ordinary car drivers, which are detected when the ordinary car drivers are in a calm state, or emotion data including the voice, expression, gesture, heart rate, and temperature of the car driver, which are detected when the car driver is in a calm state.

5. The system of claim 1, wherein the control unit comprises:
an image determining unit receiving the emotion information including the expression and gesture of the car driver to determine the emotional state of the car driver according to the current shapes of the face and mouth of the car driver;
a voice determining unit receiving the emotion information including the voice of the car driver to determine the emotional state of the car driver according to whether abuses are included in the received voice and the result of analyzing the voiceprint of the received voice;
a heart rate determining unit receiving the emotion information including the heart rate of the car driver to determine the emotional state of the car driver; and
an emotion control determining unit determining whether to control the emotion of the car driver, on the basis of the determination results of the image determining unit, the voice determining unit and the heart rate determining unit.

6. The system of claim 5, wherein the emotion controlling unit performs at least one or more of the play of therapy music, the provision of a humor, a joke or an accident possibility notice message, the spray of scent, and the generation of oxygen, for control of the emotion of the car driver.

7. The system of claim 1, wherein if the control unit determines not to control the emotion of the car driver, the control unit controls the detection unit to continue to detect the emotion information of the car driver.

8. The system of claim 1, further comprising a control panel outputting a detection request signal, used to request the detection of the emotion information for update of the reference emotion information of the car driver, according to the control of the control panel by the car driver.

9. The system of claim 8, wherein, upon receiving the detection request signal, the control unit controls the detection unit to detect the emotion information including the voice, expression, gesture, heart rate, and temperature of the car driver, and stores the received emotion information as the reference emotion information of the car driver in an update fashion.

10. The system of claim 8, wherein
the control panel outputs an emotion control exclusion signal for requesting the exclusion of the emotion control; and
upon receiving the emotion control exclusion signal during the emotion control, the control unit trains the emotional state of the car driver determined according to the emotion information of the car driver as a calm state, and adjusts the reference emotion information according to the determination result.

11. A method for controlling the emotion of a car driver, comprising:
receiving emotion information including at least one of the voice, expression, gesture, and vital data of a car driver;
comparing the received emotion information with the prestored reference emotion information of the car driver, and determining whether to control the emotion of the car driver; and
controlling the emotion of the car driver according to the determination result.

12. The method of claim 11, wherein the receiving of emotion information comprises detecting the emotion information including at lease one of the voice, expression, gesture, heart rate, and temperature of the car driver.

13. The method of claim 12, wherein the detecting of the emotion information comprises at least one of;
detecting the expression and gesture of the car driver;
detecting the voice of the car driver; and
detecting the vital data of the car driver.

14. The method of claim 11, wherein the reference emotion information is average emotion data including the voice, expression, gesture, heart rate, and temperature of ordinary car drivers, which are detected when the ordinary car drivers are in a calm state, or emotion data including the voice, expression, gesture, heart rate, and temperature of the car driver, which are detected when the car driver is in a calm state.

15. The method of claim 11, wherein the determining of whether to control the emotion of the car driver comprises:
receiving the emotion information including the expression and gesture of the car driver to determine the emotional state of the car driver according to the current shapes of the face and mouth of the car driver;
receiving the emotion information including the voice of the car driver to determine the emotional state of the car driver according to whether abuses are included in the received voice and the result of analyzing the voiceprint of the received voice;
receiving the emotion information including the heart rate of the car driver to determine the emotional state of the car driver; and
determining whether to control the emotion of the car driver, on the basis of the image determination result, the voice determination result and the heart rate determination result.

16. The method of claim 11, wherein the controlling of the emotion of the car driver comprises at least one or more of:
playing therapy music for control of the emotion of the car driver;
providing a humor, a joke or an accident possibility notice message; and
spraying scent or generating oxygen.

17. The method of claim 11, wherein the controlling of the emotion of the car driver comprises continuing to detect the emotion information of the car driver, if the emotion of the car driver is determined not to be controlled.

18. The method of claim 11, further comprising receiving a detection request signal, used to request the detection of the emotion information for update of the reference emotion information of the car driver, according to the control by the car driver.

19. The method of claim 18, wherein the controlling of the emotion of the car driver comprises:
receiving the emotion information including the voice, expression, gesture, heart rate, and temperature of the car driver upon receiving the detection request signal; and
storing the received emotion information as the reference emotion information of the car driver in an update fashion.

20. The method of claim 11, further comprising:
receiving an emotion control exclusion signal for requesting the exclusion of the emotion control during the emotion control of the car driver according to the control by the car driver;
training the emotional state of the car driver determined according to the emotion information of the car driver as a calm state; and
adjusting the reference emotion information according to the determination result.
